(19) United States
(12) Patent Application Publication
(10) Pub. No.: US 2010/0173014 A1
(43) Pub. Date: Jul. 8, 2010

(54) METHODS OF MAKING AND USING NANO SCALE PARTICLES

(75) Inventor: Michael W. Fountain, Tampa, FL (US)

Correspondence Address: PATTON BOGGS LLP, 8484 WESTPARK DRIVE, SUITE 900, MCLEAN, VA, 22102, US

(73) Assignee: Nanosolutions, LLC, Washington, DC (US)

(21) Appl. No.: 12/601,792

(22) PCT Filed: May 24, 2007
(86) PCT No.: PCT/US2008/064738

§ 371 Date: Nov. 24, 2009

Related U.S. Application Data
(60) Provisional application No. 60/924,665, filed on May 24, 2007.

(51) Int. Cl. A61K 33/14; A61K 31/194; A61K 31/661; A61K 31/522; A61P 3/02; A61P 3/12; B01J 13/02

(52) U.S. Cl. 424/679; 424/680; 514/574; 514/100; 514/263.34; 264/4.1; 977/773

(57) ABSTRACT

The instant invention discloses methods of preparing phospholipid delivery systems encapsulating one or more bio-affecting compounds, said methods comprising solubilizing a heterogeneous phospholipid mixture into a suitable organic solvent to form a concentrated formulation of phospholipids, wherein the phospholipids comprise a charged phospholipid species, and mixing the concentrated formulation with an aqueous solution comprising at least one bio-affecting compound. The instant invention also discloses methods of using a phospholipid delivery system encapsulating at least one bio-affecting compound for administration to an individual in need thereof.

### FIELD OF THE INVENTION

This invention relates to the field of phospholipids as part of bio-affecting delivery systems.

### BACKGROUND OF THE INVENTION

There have been numerous attempts in the prior art to develop lipid-based delivery systems that are capable of entrapping various materials of interest (“bio-affecting compounds”). The known methods have resulted in generally spherical delivery systems known as liposomes which are composed of a lipid bilayer having an inner space in which the entrapped material is held. These delivery systems have been formed by methods employing mechanical agitation, for example, sonication or extrusion. After lipids in organic solvents were mixed, attempts were made to dry the resulting mixture, followed by mechanical agitation and rehydration with the passenger molecule to be entrapped to encourage the lipid bilayer to enclose around the desired bio-affecting compound.

The liposomes formed by such methods were generally heterogeneous in size and difficult to sterilize for in vivo applications. The stability or shelf-life of these liposomes was often very limited. The entrapment efficiency of desired bio-affecting compounds was generally limited. The methods generally required toxic non-biocompatible solvents. The prior procedures were not applicable to aerosolization or formation of liposomes in situ. The vehicles formed by this method generally could be only sterilized by filtration, as they exhibited heat lability. Moreover, prior methodology was not acceptably adaptable to the entrapment of certain desired bio-affecting compounds.

The use of vitamin E to protect specifically against chemical-induced toxicity has been known. (Burton, et al., “Vitamin E as an antioxidant in vitro and in vivo”, Biology of vitamin E, Pitman, London (1983) London. Also see Yoshikawa and Kondo, “Role of Vitamin E in the Prevention of Hepatocellular Damage”, Vitamin E: Biochemical, Hematological, and Clinical Aspects, Lubin and Machlin, ed.; N.Y. Academy of Sci., (1982) 198-200.) Yoshikawa found no correlation between serum level of vitamin E and liver function, but did find a correlation between β-lipoprotein, a carrier of vitamin E, and liver function. Disturbance of liver function appears to arise, in such instances, from failure of effective delivery of vitamin E to the cell rather than as a result of host deficiency of vitamin E.

It has also been known that even when protection from cell injury is demonstrated using vitamin E in cell culture, a similar response often is not seen in the intact animal. The laboratory of Dr. Reed at Oregon State University has directed attention to the mechanism of protection against chemical-induced toxicity using vitamin E succinate. (See Pascoe, et al., Archives of Biochemistry and Biophysics, Vol. 253, No. 1, pp 150-158 and pp, 159-166 (1987)).

Vitamin E can prevent cell damage due to oxidative stress such as that caused by toxic injury. The protective properties of vitamin E are likely due to its role as a membrane-active antioxidant. It is believed that vitamin E, a lipid soluble vitamin, dissolves in the phospholipid environment of the membranes and can donate hydrogen to terminate the free radical-induced peroxidation of the unsaturated fatty acids of membrane phospholipids. By this mechanism, vitamin E can protect cells from free radical-induced injury.

Vitamin E deficiency results in structural and functional alterations in various tissues such as liver, brain, heart, muscle, etc. As a result, vitamin E therapies have been attempted to treat various disorders of the heart, brain, liver and muscle. Unfortunately, vitamin E therapy has produced little or no benefit in most instances. This was not surprising, since results in cultures of hepatocytes suggest that vitamin E and vitamin E acetate (VEA) were relatively inactive. Hence, it was seen that the administration of vitamin E alone as medicinal was of minimal benefit.

The need for a method of protecting liver cells from toxicity is particularly important because many medications are metabolized to toxic metabolites in the liver. A method which effectively protects the liver from medicinal-induced toxic injury would permit the use of medications that are toxic to liver tissue. An example of a compound that could be used to alleviate a disease condition but is toxic to liver tissue is tetrahydroaminoacridine (THA), a compound that has shown promise for use in treatment of Alzheimer's disease, but which is too hepatotoxic for widespread use. It has been shown that vitamin E and vitamin E succinate are useful in protecting the liver from chemical-dependent damage in vitro. However, as discussed previously, vitamin E has been found to be less useful in vivo in providing protection of the liver. (See Dogterom, et al., Biochemical Pharmacology, Vol. 37, No. 12 pp. 3211-2313 (1988)).

Attempts have been made to improve in vivo response by esterification of vitamin E. The most commonly used vitamin E esters are the acetate (VEA) and the succinate (VES) esters. (Fariss, et al., Toxicology Letters, 47 (1989) 61-75). Fariss' findings indicate that vitamin E succinate is superior to vitamin E and VEA in providing protection for cells from toxicant injury. The degree of protection seen in the cell cultures, however, has not been reflected in protection of tissue in the intact animal.

The delivery of bio-affecting agents to the site where beneficial effect is needed presents several problems. Many agents are destroyed before they reach their intended target. Furthermore, some drugs are unable to cross membrane barriers. The packaging of pharmaceutically bio-affecting agents avoids destruction in the body's environment and to effectively deliver bio-affecting agents across membrane barriers has for many years, been accomplished by the use of liposomes, microdroplets, and microcrystals. Liposomes consist of phospholipid vesicles containing water-soluble drugs (see, for example, U.S. Pat. No. 4,241,046, which is incorporated herein by reference). Other preparations such as microdroplets (see U.S. Pat. No. 4,725,442, which is incorporated herein by reference) and microcrystals (see Patent Publication WO 91/16068) have also been used.

The need for medicinals that will reduce alcohol-induced liver injury and stimulate liver cell repair is urgent, especially among women and persons of color, who respond to ingestion of alcohol with much higher levels of cirrhosis of the liver. The use of vitamin E in a form that would be effective in preventing cell damage and repairing damage to liver cells from exposure to ethanol in a form that would not be destroyed in the serum has not previously been known. The delivery of the vitamin E phosphate using phosphatidylcholine liposomes is effective in reversing damage to cells, but an effective method of manufacturing the delivery system such as that set forth in this disclosure has not been previously available.

It is the purpose of certain embodiments of this invention to provide means for protecting cells from damage and for providing means for reversing cell damage by administration of vitamin E phosphate in the form of liposomes, particularly those prepared with phosphatidylcholine and most preferably using polyenylphosphatidylcholine (PPC).

It is our present understanding that vitamin E phosphate protects cells from the effects of oxidative stress and enhances the repairing process in damaged cells. The vitamin E phosphate in phosphatidylcholine, especially polyenylphosphatidylcholine (PPC), liposomes is particularly useful for protecting the tissue or ameliorating cell damage in the intact animal. A route of administering for effecting protection of liver tissue is intra-peritoneal injection or infusion; however, oral administration is more preferred when possible. The carrier used in the vitamin E phosphate/phosphatidylcholine liposome-containing composition and the mode of administration will depend on the target organ. The phosphatidylcholine both protects the vitamin E phosphate from inactivation in the serum and enhances the cellular repairing properties of the composition. The vitamin E phosphate/phosphatidylcholine liposomes provide benefits not available when administering the two components separately, even though they may be administered simultaneously. Because the growth of liver cells in tissue culture is very useful for research, for diagnostic purposes and for production of products of the liver in vitro, the use of the vitamin E phosphate/phosphatidylcholine in tissue culture is also an important embodiment of this invention.

### SUMMARY OF THE INVENTION

The present invention relates to phospholipid delivery systems and methods of preparing phospholipid delivery systems for use in encapsulating bio-affecting compounds for administration to subjects in need thereof. In many embodiments, the present invention relates to a nanoscale particle (NSP) complex comprising a phospholipid delivery system and at least one bio-affecting compound for administration to a subject. Phosphatidylcholine—especially polyenylphosphatidylcholine (PPC)— liposomes with and without vitamin E phosphate are particularly preferred. The complexes of the present invention are in formulations where some or nearly all of the bio-affecting compounds are encapsulated.

Certain embodiments of the present invention exhibit high entrapment efficiencies. Thus, entrapment efficiencies of greater than 60% are preferred, more preferable efficiencies of 75 or 80% are desired; even greater entrapment efficiencies of 85, 90, 95 or even higher percents are contemplated.

Certain embodiments of the present invention relate to a concentrated intermediary shelf-stable NSP complex and methods of making for ready dilution to form an NSP complex for administration to a subject in need thereof.

Also disclosed are methods of preparing NSP complexes to mask the taste of bio-affecting compounds when the NSP complexes are administered orally. These NSP complexes can be formed in an intermediate product that can be stored for rapid dilution to make a still or carbonated drink.

Embodiments of the present invention also relates to NSP complexes comprising encapsulated therapeutic compounds for administration to a subject in need thereof. Certain NSP complexes of the present invention assist in repair of cellular damage, especially the type of damage often associated with aging. The complexes can be used to protect both inside and outside the cell or body. The complexes can repair and protect against damage as well as induce the body's own repair mechanism. Embodiments can be used as general immunity boosters, avoid or inhibit memory loss and provide a number of pharmaceutical applications. Certain improved embodiments, preferably those using polyenylphosphatidylcholine (PPC) provide unexpectedly superior results for repair and healing. Improved embodiments utilizing glutathione are particularly beneficial in improving certain health effects.

Phosphatidylcholine—especially polyenylphosphatidylcholine (PPC)— liposomes with and without vitamin E phosphate may be added to foods or beverages to supplement the diet or given orally in tablet or capsular form to protect from the damaging effects of oxidative stress and to assist in cell repair functions. Such phosphatidylcholine complexes can also be used as dietary supplement either alone or in conjunction with other dietary enhancing components.

Because many otherwise useful drugs are not given because of their effect on liver cells, the use of vitamin E phosphate/phosphatidylcholine liposomes given in conjunction with such drugs can provide useful benefits. The NSP of embodiment of the present invention make this possible. Administration with vitamin E phosphate/phosphatidylcholine to protect the liver may render such drugs far less objectionable as long-term treatments.

Incorporation of membrane proteins into the bilayer of the liposomes and incorporation of proteins or peptides into the liquid are also contemplated embodiments.

### DETAILED DESCRIPTION

The present invention pertains generally to the production and use of phospholipid delivery systems for use in making (i) nanoscale particles (NSP) complexes comprising phospholipid delivery systems and at least one bio-affecting compound encapsulated in the phospholipid delivery system; (ii) concentrated shelf-stable NSP complexes comprising phospholipid delivery systems and at least one bio-affecting compound encapsulated by the phospholipid delivery system; and/or (iii) wherein the NSP complexes are designed to mask or partly mask the taste of at least one bio-affecting compound.

The vitamin E phosphate/phosphatidylcholine liposomes can, in accordance with the teachings herein, be added to solutions used for storage and transport of tissues for transplant. One of the major problems in the transportation of organs is the damage to cells between the time the organ is harvested and the time the organ is connected to the recipient's blood supply. The use of vitamin E phosphate/phosphatidylcholine liposomes to prevent tissue damage could greatly assist in improving the efficacy of such transplants. The concentration of the vitamin E phosphate/phosphatidylcholine liposomes can vary greatly. For example, concentrations of 1 μM to 1000 μM would be appropriate. A preferred concentration is 10 μM to 100 μM. The vitamin E phosphate in the vitamin E phosphate/phosphatidylcholine liposomes may be in the form of one of the soluble salts, such as the sodium or potassium salts, in isotonic solution. The use of vitamin E phosphate/phosphatidylcholine liposomes as an additive to such solution for storage and transport would be useful with any tissue for transplant, such as heart, liver, muscle (including heart muscle), lung, kidney tissue. Many of the chemical compositions of phosphatidylcholine liposomes are taught in U.S. application Ser. Nos. 09/670,346 and 11/070,738 to Lamb, which are incorporated herein in their entirety.

The use of polyenylphosphatidylcholine (PPC) as the former for the phospholipid delivery system, as used herein, provides exceptional results. As described above, vitamin E phosphate (VEP) can be used to inhibit tissue damage in transplant organs/tissues. The use of PPC as the phospholipid delivery system for the VEP yields unexpected results, whereby the VEP/PPC combination is almost five times more effective in reducing particular adverse affects of such compounds as ethanol on liver cells for example. In fact, in experiments using the VEP/PPC, the VEP/PPC combination essentially blocked the adverse cellular effects of ethanol.

As an example of the effectiveness of using the PPC, the following experiments were conducted. Cultured liver cells were incubated for 24 hours with 100 mM ethanol in the presence of (1) water, (2) VEP/EPC (egg phosphatidylcholine), or (3) VEP/PPC. Agent-dependent alterations in cell function were determined by measuring phosphatidylcholine biosynthesis. Similar results were obtained in the three separate preparations of cultured cells, all of which are reflected in the Table 1 set forth below.

Control cells are expressed as 100%, meaning full cellular function. A reduction below 100% represents a decrease in cell function. Cells exposed to 100 mM ethanol for 24 hours exhibited a significant (p<0.01) reduction in cell function, down 63% from control cells.

Cells incubated with 100 mM ethanol for 24 hours in the presence of VEP/EPC (15 μM EPC) showed a significant (p<0.01) reduction in the adverse cellular effects of ethanol, as cellular function was only decreased by 33%.

Surprisingly, however, cell incubated with 100 mM ethanol for 24 hours in the presence of VEP/PPC (15 μM VEP and 30 μM PPC) only displayed a 7% reduction in cell function. This demonstrates that VEP/PPC was significantly (p<0.01) better in reducing the adverse cellular effects of ethanol than VEP/EPC. In fact, VEP/PPC was found to be almost five times more effective than VEP/EPC in reducing the adverse effects of ethanol on cells.

The results are displayed in the following Table 1:

TABLE 1
% of PC biosynthesis in cells incubated with
the listed Addition, as compared to Control

| Additions | % Control ± SEM |
|---|---|
| None | 100 ± 2 |
| Ethanol | 37 ± 1* |
| Ethanol + VEP/EPC | 67 ± 4** |
| Ethanol + VEP/PPC | 93 ± 3*** |

*Level of significance from control (none) is p < 0.01
**Level of significance from Ethanol is p < 0.01
***Level of significance from Ethanol + VEP/EPC is p < 0.01

These results demonstrate that the VEP/PPC has a surprising superiority at protecting cells from injury. These results also demonstrate that PPC is unexpectedly superior to saturated forms of phosphatidylcholine, such as egg phosphatidylcholine.

It should be appreciated that the effects of PPC, as used in the present invention, possess surprising delivery activity in relation to the bio-affecting compounds and solvents as described herein, whether VEP is or is not part of the composition manufactured and/or administered. In compositions manufactured and/or administered within the scope of this invention, the use of VEP/PPC in compositions with at least one other bio-affecting compound results in a surprisingly potent cytoprotective agent, thereby reducing and/or inhibiting oxidative stresses on cells.

Phospholipid Delivery System

In one embodiment within the scope of this invention, phospholipid delivery systems can be made by solubilizing a heterogeneous phospholipid mixture into a suitable organic solvent to form a concentrated formulation of phospholipids. Preferably the heterogeneous phospholipid mixture is rich in polyunsaturated [polyenyl phospholipids] fatty acids. Examples of phosphatides to be used within the scope of the invention include but are not limited to, phosphatidylcholine (such as polyenylphosphatidylcholine), phophatidylethanolamine, phosphatidic acid and phosphatidylinositol. Within the phospholipid and solvent solution there can be at least one species of charged phospholipids, wherein the phospholipid is preferably charged at a pH of 7, preferably a negatively charged phospholipid. While not wishing to be constrained by any current theory of action, it is presently believed that the charged phospholipids aid in keeping components separate in the formulations. The charged phospholipids are also effective in maintaining size of the delivery systems through judicious choice of the appropriate concentration in the organic solvent used, for example ethanol. By providing a charged surface for controlled size, we have found it is possible to avoid the natural tendency of the components to stick together. Such adhesion can lead to larger liposomes through fusion, or can simply lead to a larger effective size due to clumping of the vesicles. This is a problem due to the increase in size and lack of uniformity; thus effecting delivery. Avoiding the adhesion leads to smaller population size distributions of the phospholipid delivery systems and/or greater uniformity for materials used within the scope of the present invention.

One of the most preferred phospholipids of the present invention is the polyenylphosphatidylcholine (PPC) from soy lecithin. Unsaturated phosphatidylcholine is commonly extracted from soy lecithin. It contains choline and omega-6-unsaturated fatty acid (linoleic acid) plus smaller quantities of omega-3-fatty acids (gamma-linolenic acid), all essential for human life. The human body is not able to synthesize these substances. A typical fatty acid composition of soy phosphatidylcholine, as instantly claimed as polyenylphosphatidylcholine (PPC), comprises: 10.5% oleic acid; 66.5% linoleic acid; and 5.7% linolenic acid. Therefore, 82.7% of the fatty acids in soy phosphatidylcholine are in the unsaturated form, meaning these phosphatidylcholines are in the ‘polyenyl’ form either through one of the fatty acids or through a combination of the fatty acids. While the soy PPC is particularly preferred because of its excellent results and ease of manufacture and availability, PPC from any source is also preferred.

The appropriate solvent is selected from those able to solubilize the phospholipid materials. Generally, the solvent is a low molecular weight hydrocarbon such as ethanol, propanol, or chloroform, and the like. In addition, the solvent is preferably chosen to be appropriate for the particular intended use of the phospholipid delivery system. The solvent is preferably utilizable without causing toxicity in that use and generally should be biocompatible and readily miscible. Mixtures of solvents may be appropriate in some circumstances.

The term “charged phospholipid” means a natural or synthetic phospholipid which is electrically charged at neutral pH. A “negatively charged phospholipid” (also known as an “anionic phospholipid”)) has a negative charge at neutral pH. A “positively charged phospholipid” (also known as a “cationic phospholipid”) has a positive charge at neutral pH.

Those of skill in the art will appreciate the properties of desired charged phospholipids, preferably a negatively charged phospholipid. Examples of negatively charged phospholipids include, but are not limited to, phosphatidic acid, phosphatidylserine, and fatty acids of polyenylphosphatidylcholine. Without tending to be bound by any theory or theories of operation, it is contemplated that such negatively charged lipids provide added stability by counteracting the tendency of phospholipid delivery systems to rupture by fusing together. Thus, the negatively charged lipids may act to establish a uniform negatively charged layer on the outer surface of the delivery system, which will be repulsed by a similarly charged outer layer on other delivery systems which are proximate thereto. In this way, the delivery systems may be less prone to come into touching proximity with each other; thus, avoiding a rupture of the membrane or skin of the respective delivery system and consolidation of the contacting delivery systems into a single, larger delivery system. A continuation of this process of consolidation will, of course, lead to significant degradation of the delivery systems to be employed in the scope of this invention.

In another aspect, this invention relates to positively charged (cationic) phospholipid compositions, and the use of these compositions to manage size of the phospholipid delivery systems or NSP complexes described herein. One of skill in the art will recognize the applicability of positively charged lipid compositions for use within the scope of the instant invention. Ideally, positively charged phospholipids for use in the instant invention will be selected so that it is biocompatible without causing any deleterious effects in vivo. In another aspect, one of skill in the art will readily recognize the use of neutral phospholipids within the scope of the present invention as it pertains to phospholipid delivery systems, as well as the nanoscale particle complexes described herein.

An optically clear solution of phospholipids in organic solvent can be prepared by solubilizing a heterogeneous phospholipid mixture containing soy phospholipids and phosphatidic acid, for example, in an appropriate ratio in ethanol. As a non-limiting example, the phosphatides to be used within the scope of the instant invention can be purified soybean phospholipids. A phospholipid delivery system formed by this method is characterized by having optical clarity at room temperature and being monophasic at room temperature. Thus, in the present method, phospholipids are dissolved in an organic solvent appropriate to affect the complete dissolution thereof and which is compatible with the desired application. This solution of phospholipid delivery systems can also be used as a stock solution for end or middle users including but not limited to hospitals, physicians, pharmaceutical manufacturers, and sports athletes, for examples.

Nanoscale Particle (NSP) Complex

In one embodiment of the instant invention, the phospholipid delivery system can be used in preparation of formulations comprising in part an at least one bio-affecting compound for administration to a subject in need thereof. Bio-affecting compounds can be solubilized, in water for example, to produce a concentrated aqueous solution. Such compounds may also be solubilized in an organic solution for creation of the vesicles. The concentrated aqueous solution can then be combined with the phospholipid delivery system to form a nanoscale particle (NSP) complex in a concentrated shelf-stable formulation or in a diluted ready to administer formulation. The shelf-stable NSP complex solution has the attributes of controlled size by lipid composition and applicable solvent giving the ability to convert to a final administrable product. Also, this approach allows for high loading capacity of the phospholipid delivery system. The process yields an optically clear solution which is highly desired and may also affect the efficacy of certain embodiments of present invention. By using negatively charged phospholipids with an appropriate biocompatible solvent, such as ethanol, the requirement for energy agitation such as shaking or sonication is reduced or eliminated, wherein now the system is driven by the negative phospholipids and solvent. This allows for the inherent characteristic of rehydration. This also provides a quality size that is uniform across the shelf-stable NSP complex solution and the final administrable product. It is presently believed that the resulting liposomes or NSPs have a far greater percentage of unilamellar complexes than when generated utilizing great amounts of shaking or sonication; thus, providing a quality size for absorption into the cell or body of an individual in need thereof. It is presently believed that these unilamellar complexes are better vehicles for absorption into the cell or body than the heterogeneous uni—and/or multi-lamellar distributions created with sonication or shaking. While not wishing to be constrained by any particular theory of action, it is also believed that the NSP complexes, because of their small size, pass easily from the stomach to the small intestine and are not completely blocked by the valve that normally restricts passage into the intestine. One advantage of the NSP complexes of the phospholipid delivery systems used herein is that the uniform smaller size due to the negatively charged phospholipids yields an unexpected effectiveness in delivery of the bio-affecting compounds. It will be understood by one of skill in the art that while the mostly unilamellar NSP complexes are uniformly small in size, that size may vary depending on the phospholipids and the bio-affecting compounds chosen for delivery.

Both water soluble and/or lipid soluble bio-affecting compounds may be easily incorporated into the finished NSP complex of the shelf-stable complex or the administrable complex. For water soluble bio-affecting compounds, solubility is defined as solubility in pure water or any aqueous phase such as a salt solution. The volume of phospholipid delivery system solution to be added to the bio-affecting compound solution depends upon the solubility of the desired bio-affecting compounds as well as the intended concentration within the liposome. The volume of phospholipid delivery system solution required increases with decreasing solubility of the desired bio-affecting compounds, and can be readily determined through routine experimentation. The bio-affecting compounds may be generally any material capable of being retained by or in a formed bilayer or associated with that bilayer of the phospholipid delivery system. For example, the bio-affecting compounds can be lipophilic; however, hydrophilic bio-affecting compounds may also be utilized if they are capable of forming an association with the bilayer of the phospholipid delivery system.

One of skill in the art will understand that the NSP complex is at least partially an encapsulating complex, wherein at least a percent range of the at least one bio-affecting compounds is encapsulated within the phospholipid delivery systems to be used in the instant invention. Due to the chemical characteristics of the components involved and the mixture amounts desired for formation of a particular NSP complex, a percent range of bio-affecting compounds will remain in the solution wherein they are not encapsulated by the phospholipid delivery systems. Within the scope of the invention, the percent of the at least one bio-affecting compounds that are not encapsulated by the methods herein disclosed is less than 50%, preferably less than 20%, more preferable less than 10% and most preferably less than 5%. Those percentages of at least one bio-affecting compound that are not encapsulated can be removed, where desired, by those methods generally known in the art. As an example of such methods the non-encapsulated percentages of at least one bio-affecting compound could be removed by exclusion chromatography. It should be understood by one of skill in the art that techniques such as filtration, especially pressure filtration, centrifugation and precipitation may be employed to separate the encapsulated bio-affecting compounds from the bio-affecting compounds not encapsulated. This would be desired in situations where taste of the administrable NSP complex is a concern and non-encapsulated bio-affecting compound is present after creation of the vesicle.

Although separation and purification may be desired in particular situations, it is intended that the products and methods of the instant invention need not be subjected to separation and purification in most circumstances. While it is understood that the percentages of non-encapsulated bio-affecting compounds will vary depending on the components used herein, it should be readily recognized that the presence of excess is acceptable or desired in some situations. As a non-limiting example, excess bio-affecting compounds in at least some situations will be readily utilized in vivo by the natural processes of the body receiving the compounds. In these situations, the minimum excess will not affect the taste masking aspects of the invention described herein.

Those of skill in the art will appreciate the properties of desired bio-affecting compounds encompassed by the instant invention. Non-limiting examples of bio-affecting compounds that can be utilized in the instant invention alone or in combination are caffeine, desired vitamins, minerals and salts, therapeutic drugs or pro-drugs, and other desired bio-affecting compounds.

The concentrated shelf-stable NSP complex formulation can be produced at room temperature with mixing. Alternatively, higher and lower temperatures can be utilized. Production of the shelf-stable NSP complex formulation can be achieved by mixing the phospholipid delivery system solution into a concentrated aqueous solution of a suitable bio-affecting compound. At room temperature, this will generally result in an intermediary NSP complex wherein the sizes can range from about 180 to 250 nm, for example. By employing negatively charged phospholipid species, the size can be controlled and separation of the complexes can be maintained. These complexes have up to two bilayer configurations, with the understanding that the goal of the present invention is a primarily unilamellar bilayer system and that much of the material formed is unilamellar.

In one embodiment of the instant invention, a ready to administer formulation is prepared comprising the NSP complex. An administrable NSP complex can be prepared from a concentrated shelf-stable NSP complex as described herein, by diluting the concentrated NSP complex formulation to the desired concentration suitable for administration. For administration, the NSP complex would comprise a desired at least one bio-affecting compound. Alternatively, an administrable NSP complex can be prepared by mixing a desired phospholipid delivery system described herein with a diluted aqueous solution comprising an at least one desired bio-affecting compound.

As an illustration of an administrable NSP complex, a shelf-stable NSP complex may be diluted in a suitable aqueous solution. Examples of dilution ranges can be between about 1:10 and about 1:100. The size range for administrable NSP complexes can be in a range of about 100 to 180 nm. These complexes can have 1-2 bilayers with the understanding that the object of the present invention is a primarily unilamellar bilayer system.

The preparation of an intermediate vesicle formulation can also permit easy carbonation of the resulting beverage. This carbonation not only increase the consumer appeal of the product, but also permits simplified protection from spoilage. Alternatively, other well known methods can be used to retard spoilage.

The traditional method of pasteurization was vat pasteurization, which involved heating the liquid ingredients in a large vat or tank for at least 30 minutes. Variations on the traditional pasteurization methods have been developed, such as, high temperature short time (HTST) pasteurization, ultra pasteurization (UP) processing, and ultra high temperature (UHT) pasteurization. These variations on the traditional pasteurization method use higher temperatures for shorter times, and may result in increased shelf lives without refrigeration. Regardless of the pasteurization method used, however, stabilizers and preservatives may often be needed to improve the stability of the products.

Thermal processing by any pasteurization method may have detrimental effects on the organoleptic and nutritional properties of treated materials. Thus, there can be a need for more non-thermal methods of extending shelf life that will not significantly decrease or alter the organoleptic and nutritional properties of the treated materials.

One alternative to pasteurization is high pressure processing (HPP), which may be especially suited to high acid content foods. HPP is a food processing method where food products may be exposed to elevated pressures, in the presence or absence of heat, to inactivate microorganisms. HPP may also be known as high hydrostatic pressure processing (HPP) and ultra high-pressure processing (UHP).

Non-thermal HPP may be used to extend the shelf life of products without detrimentally altering the organoleptic and nutritional properties of these products. Non-thermal HPP may eliminate thermal degradation, and may allow for the preservation of “fresh” characteristics of foods. Shelf lives similar to those of pasteurized products may be achieved from HPP.

HPP of a product may be achieved by placing the product in a container within a water (or other pressure-transmitting fluid) filled pressure vessel, closing the vessel, and increasing the pressure exerted upon the container by pumping more water into the pressure vessel by way of an external pressure intensifier. The elevated pressure may be held for a specific period of time, then it may be decreased. Pressure levels of about 600 MPa at 25° C. may typically be enough to inactivate vegetative forms of microorganisms, such as non-spore forming pathogens, vegetative bacteria, yeast and molds.

HPP is explained in more detail in U.S. Pat. No. 6,635,223 B2 to Maerz, issued Oct. 21, 2003, entitled “Method for inactivating microorganisms using high pressure processing”, wherein a method for inactivating microorganisms in a product using high pressure processing is disclosed. The method involves the steps of packing the product in a flexible container, heating the product to a pre-pressurized temperature, subjecting the product to a pressure at a pressurized temperature for a time period; and reducing the pressure after that time period. The method may also further comprise an additional step of subjecting the product to a predetermined amount of oxygen for a time interval. These methods may be applied to food, cosmetic or pharmaceutical products.

Carbon dioxide (CO₂) is also known to have antimicrobial properties. CO₂ results in minimal harm in foods; therefore, it is a suitable agent for inhibiting food spoilage microorganisms. Currently, there are at least three general mechanisms known by which CO₂ inhibits microorganisms. These mechanisms, outlined briefly below, are discussed in more detail in an article by J. H. Hotchkiss et al., in Comprehensive Reviews in Food Science and Food Safety 2006; 5: 158-168, “addressing the addition of carbon dioxide to products to improve quality.”

The first mechanism by which CO₂ may inhibit microbial growth is simply by the displacement of O₂ by CO₂. The second mechanism by which CO₂ may inhibit microbial growth is by lowering the pH of the food by the dissolution of CO₂ and formation of carbonic acid in the aqueous phase of the food by the following equilibrium reactions: H₂O+CO₂ [symbol] H₂CO₃ [symbol] H⁺+HCO₃⁻ [symbol] 2H⁺+CO₃²⁻. The third mechanism by which CO₂ may inhibit microbial growth is by a direct effect of CO₂ on the metabolism of microorganisms.

The third mechanism, the direct antimicrobial effect of CO₂ on the metabolism of microorganisms, may be the result of changes in membrane fluidity due to CO₂ dissolution, reductions in intracellular pH, and direct inhibition of metabolic pathways, including decarboxylation reactions and DNA replication. CO₂ is quite lipophilic, which may allow for it to concentrate within the lipid membrane of bacteria, or to pass through the lipid membrane and to concentrate within the bacterial cell lowering intracellular pH. CO₂ may also interfere directly with required enzymatic processes within microorganisms, such as gene expression.

The interaction between HPP and CO₂ and their effects on food spoilage enzymes and microorganisms were described by Corwin and Shellhammer in Journal of Food Science 2002; 67: 697-701, entitled “Combined carbon dioxide and high pressure inactivation of pectin methylesterase, polyphenol oxidase, Lactobacillus plantarum and Escherichia coli.” The enzymes studied were pectin methylesterase (PME) and polyphenol oxidase (PPO) and the microorganisms studied were Lactobacillus plantarum ATCC 8014 (L. plantarum), an acid tolerant, lactic acid producing, non-spore forming, Gram positive bacterium, and Escherichia coli K12 (E. coli), an acid sensitive, non-spore forming, Gram negative bacterium. The objective of the study was to determine the effect of CO₂ on increasing the efficacy of pressure processing to inactivate enzymes and microorganisms. CO₂ was added at approximately 0.2 molar % to solutions processed at 500 to 800 MPa in order to further inactivate PME, PPO, L. plantarum, and E. coli. A significant interaction was found between CO₂ and pressure at 25° C. and 50° C. for PME and PPO, respectively. Activity of PPO was said to be decreased by CO₂ at all pressure treatments. Survival of L. plantarum was said to be decreased by the addition of CO₂ at all pressures and the combination of CO₂ and high pressure had a significant interaction. CO₂ was said not to have a significant effect on the survival of E. coli under pressure.

The methods disclosed herein are for preparing products for administration to subjects in need thereof, or for preparing products to use in the preparation of administrable products. Examples of administrable products of the present invention include but are not limited to oral hydration products, caffeine products, and therapeutic products. Furthermore, the products within the scope of the present invention and the methods of preparing those products are designed to mask an undesired taste or flavor of an at least one bio-affecting compound desired for delivery to a subject in need thereof.

Phospholipid delivery systems and/or NSP complexes, concentrated or administrable, within the scope of the instant invention may be evaluated by utilizing a light scattering technique to determine the presence of delivery systems and/or the NSP complexes. This technique can also be used to estimate the size of the phospholipid delivery systems and/or NSP complexes. Various instruments are commercially available for the sizing and counting of delivery systems or NSP complexes. Particle analyzers are an example of such instruments employed to measure submicron particles. Phospholipid delivery systems and NSP complexes may also be estimated using standard column chromatography techniques. The phospholipid delivery systems and NSP complexes have also been analyzed by testing the efficacy of the phospholipid delivery systems and NSP complexes over standard commercial preparations of the at least one bio-affecting compounds. The phospholipid delivery systems and NSP complexes were found to have successfully encapsulated the at least one bio-affecting compound of interest by utilizing standard tests for the efficacy of the at least one bio-affecting compounds.

It was found that phospholipid delivery systems and NSP complexes of the instant invention exhibit substantial size homogeneity. The size is believed to be dependent on the at least one bio-affecting compound and identity of the phospholipid materials utilized, but it has been demonstrated that within one preparation of phospholipid delivery systems, the size range is very compact. The size is also believed to be dependent upon the ionic strength of the aqueous phase used in the creation of the liposomes. This characteristic is believed to be important in several applications of phospholipid delivery systems and NSP complexes including in vivo delivery of oral hydration material and other bio-affecting compounds. Size can also be affected by homogenization or sonication of the intermediate product.

The phospholipid delivery systems and NSP complexes have also been tested to be stable to flash pasteurization, which widens their utility for uses where sterility is required.

The invention may be better understood by the following non-limiting examples that are intended to be illustrative thereof.

### Example 1

### Preparation of Oral Hydration Product

An optically clear, solubilized solution of heterogeneous phospholipids can be prepared by solubilizing a phospholipid mixture containing phosphatidylcholine, phosphatidic acid and ethanol, for example. This provides a desired phospholipid delivery system for use in making the NSP complex for an oral hydration product.

As an example of bio-affecting compounds that can be utilized in a desired oral hydration product within the scope of the invention, include but are not limited to alone or in combination, sodium chloride (NaCl), potassium chloride (KCl), trisodium citrate and Vitamin E Phosphate.

In one embodiment of an oral hydration product, an optically clear solubilized solution of mixed phospholipids was prepared by solubilizing a soybean phospholipid mixture (American Lecithin Company New York, N.Y.) containing 85 mg of phosphatidylcholine, 7 mg of phosphatidic acid and 100 ml of ethanol. A concentrated solution of bio-affecting compounds containing 62 g of NaCl, 22 g of KCl, 45 g of Trisodium Citrate, 52 mg Vitamin E Phosphate (Intezyne, Tampa, Fla.) and 948 ml of distilled water was prepared by first solubilizing Vitamin E Phosphate in distilled water while stirring. Following the solubilization of the Vitamin E Phosphate, NaCl, KCl and Trisodium Citrate were sequentially added until each had been solubilized into the aqueous mixture. To the concentrated mixture of bio-affecting compounds in distilled water was added 52 ml of the optically clear solution of phospholipids. Production of the nanoscale phospholipid and bio-affecting compound intermediary complexes was accomplished by gentle mixing at room temperature. The size of the nanoscale intermediary complexes was 190 nm. Production of finished nanoscale particles containing bio-affecting compounds was accomplished by dilution of nanoscale intermediary complexes 1:75 into distilled water while stirring. The average size of the nanoscale particle was 130 nm.

### Example 2

### Masking the Taste of Bio-Affecting Compounds Using the NSP Complex

Administrable products were evaluated by oral administration for salty flavor. Subjects were administered, for example, a preparation of Example 1. In one embodiment, the NSP complex comprised NaCl and KCl as bio-affecting compounds that were encapsulated in the NSP complex. It was discovered that the administrable NPS complexes effectively masked the taste of salts in the preparation.

### Example 3

### Preparation of Caffeine Products

An optically clear, solubilized solution of mixed phospholipids was prepared by solubilizing a soybean phospholipid mixture (American Lecithin Company New York, N.Y.) containing 85 mg of phophatidylcholine, 7 mg of phophatidic acid and 100 ml of ethanol. A concentrated solution of bio-affecting compound Caffeine was prepared by dissolving 50 mg of Caffeine into 10 ml of distilled water. To the concentrated mixture of bio-affecting compound in distilled water was added 1 ml of the optically clear solution of phospholipids. Production of the nanoscale phospholipid and bio-affecting compound intermediary complexes was accomplished by mixing at room temperature. The size of the nanoscale intermediary complexes was 200 nm. Production of finished nanoscale particles containing bio-affecting compounds was accomplished by dilution of nanoscale intermediary complexes 1:100 into distilled water while stirring. The average size of the nanoscale particle was 160 nm.

### Example 4

### Masking the Taste of the Caffeine NSP Complex

Caffeine, especially in concentrated solutions, has an extremely bitter taste and is used in many caffeine containing beverages without suitable masking agents. To avoid this bitter taste, an encapsulation technique of the present invention was tested.

Administrable caffeine NSP complex products were evaluated by oral administration and testing for a metallic caffeine taste. Subjects were administered, for example, a preparation of Example 3. In one embodiment, the NSP complex comprised caffeine as an bio-affecting compound that was encapsulated in the NSP complex. The administrable NPS complex effectively masked the bitter metallic taste of caffeine in the preparation.

One of skill in the art will readily see the applicability of the products and methods of the instant invention in forming dried or dehydrated forms for packaging and transport. The products of the instant invention may be dried, such as dehydration. Spray drying or fluid bed drying are examples of drying techniques commonly known and easily applied to this technology. The dehydrated forms can be prepackaged and sold and/or transported easily in large quantities or smaller quantities. As non-limiting examples, this aspect can occur in bulk or by spraying the surface of an object and dehydrating the system. Dried or dehydrated forms have the added benefits of convenient shipping or transport. Furthermore, the dried and/or dehydrated forms are readily reconstituted to the desired dilutions for a range of uses. Due to the NSP complex formulations, the compositions do not require added bulking agents or stabilizers to reconstitute an administrable formulation. Drying and/or dehydrating the system can produce small pellet or granular forms of the NSP complexes. These forms are readily reconstituted for administration. These methods and products may be employed in devices such as tampons, topical compounds, bandages or wraps, and can be used in preparation and use in parenteral, intravenous, intramuscular, or subcutaneous types of injections.

In another embodiment, the present invention can be used in larger scale situations such as water delivery tanks. The products and methods of the instant invention provide easy use in hydration by being useful in water coolers, tanks, basins, or the like for large scale administration and delivery to many individuals. The dehydrated forms, for example, can be readily mixed into a large water or consumable liquids receptacle from which individuals may draw the quantity of fluids desired or necessary. This can be achieved through the mixing of dried or dehydrated forms of the concentrate or by the concentrated NSP complex, for example. For example, a 75 times concentration of dehydrated NSP complex form can be reconstituted to form in excess of 1665 liters of ready to administer or consumable solution. As one of skill in the art will readily see, the methods and products within the scope of the instant invention are only limited by the resources available regarding the large scale mixing. In other words, if resources permit, a large scale of 100,000 liters of ready to administer NSP complex formulation can be prepared for example.

In another embodiment, the products and methods described herein may be used for carbonated drinks, mixed drinks, mouthwashes, or cocktails. For example, carbonated drinks, such as sodas and seltzers, can employ the products of the instant invention by incorporating the NSP complex as described herein. The carbonation does not disrupt or rupture the integrity of the NSP complex system. Mixed alcoholic beverages may employ the products as described herein. The alcohol in mixed drinks and cocktails does not disrupt or rupture the NSP complex system. Whether used directly from the shelf-stable intermediate NSP complex or from a dried, dehydrated formulation, the level of residual solvent used therein is insignificant and would serve or be labeled as no more than a preservative. Again, the scale of production is only limited by the resources available at the time of manufacture. This aspect can occur on an individual drink scale, or on a mass production line scale, wherein large receptacles, vats or cauldrons are utilized, such as in the beverage industry.

In regards to all products and methods of the instant invention, the NSP complexes, for delivery or administration to individuals in need thereof, mask the taste of those compounds that are part of the NSP complex.

In another embodiment, the phospholipid delivery systems and/or NSP complex formulation may used in the manufacture of hygiene products, such as douches, mouth washes/rinses, toothpastes, and sanitary napkins. The NSP complexes of the instant invention can be employed in situations where antimicrobials are desired for delivery to the body via oral washes/rinses or toothpastes, or vaginal applications through a douche or sanitary napkin. Furthermore, oral washes/rinses or toothpastes may be used with the NSP complexes for delivery of the bio-affecting compounds, such as salts including sodium, potassium and fluoride, for example. Sanitary napkins and/or douches may be used with the NSP complexes for delivery of compounds that aid in odor control, for example.

The liposomes of the invention also show a surprising ability to be absorbed into the bloodstream through the mucosal membranes of the mouth, for example. Thus, some medicaments can be administered by placing the material in the mouth, even if it is not swallowed. In addition, the liposomes can be either applied topically or applied to clothing. In this manner, liposomes can be designed so that perspiration, or other bodily fluids, actually triggers the release of the encapsulated material. This would allow their usage, without limitation, as an antiperspirant/deodorant, for example.

Medical Applications

The products and methods of the instant invention can be employed in the treatment of various disorders, especially wherein hydration or fluid volume levels are important in the maintenance, treatment or alleviation of such disorders or symptoms. The NSP complexes of the present invention are ideal for delivery of a balanced composition of suitable salts, nutritional supplements, vitamins and/or natural herbs and/or extracts for aiding in fluid volume control and/or treatment or alleviation of related disorders or symptoms of such disorders. The NSP complexes aid in masking the taste of desired compounds for oral delivery. These products and methods are aid in effective delivery of the desired components. The products and methods of the instant invention allow for micro- or nano-encapsulation of suitable salts in an effective manner to treat and/or alleviate the disorders and/or related symptoms of such disorders. The unilamellar nature of the created material also facilitates uptake by or delivery to cells and organelles of the body. As non-limiting examples of such disorders, the instant invention can be effective in the treatment and/or alleviation of symptoms associated with chronic fatigue syndrome and vasodepressor carotid sinus syndrome, for examples.

Chronic Fatigue Syndrome (CFS)

Chronic fatigue syndrome, or CFS, is a debilitating and complex disorder characterized by profound fatigue that is not improved by bed rest and that may be worsened by physical or mental activity. Persons with CFS most often function at a substantially lower level of activity than they were capable of before the onset of illness. In addition to these key defining characteristics, patients report various nonspecific symptoms, including weakness, muscle pain, impaired memory and/or mental concentration, insomnia, and post-exertional fatigue lasting more than 24 hours. In some cases, CFS can persist for years. The cause or causes of CFS have not been identified and no specific diagnostic tests are available. Moreover, since many illnesses have incapacitating fatigue as a symptom, care must be taken to exclude other known and often treatable conditions before a diagnosis of CFS is made.

A number of illnesses have been described that have a similar spectrum of symptoms to CFS. These include fibromyalgia syndrome, myalgic encephalomyelitis, neurasthenia, multiple chemical sensitivities, and chronic mononucleosis. Although these illnesses may present with a primary symptom other than fatigue, chronic fatigue is commonly associated with all of them.

In addition to the eight primary defining symptoms of CFS, a number of other symptoms have been reported by some CFS patients. The frequencies of occurrence of these symptoms vary from 20% to 50% among CFS patients. They include abdominal pain, alcohol intolerance, bloating, chest pain, chronic cough, diarrhea, dizziness, dry eyes or mouth, earaches, irregular heartbeat, jaw pain, morning stiffness, nausea, night sweats, psychological problems (depression, irritability, anxiety, panic attacks), shortness of breath, skin sensations, tingling sensations, and weight loss.

Chronic fatigue syndrome (CFS) affects more than one million people in the United States. There are tens of millions of people with similar fatiguing illnesses who do not fully meet the strict research definition of CFS. People of every age, gender, ethnicity and socioeconomic group can have CFS and possess the risk factors associated with CFS. CFS affects women at four times the rate of men. Research indicates that CFS is most common in people in their 40s and 50s. Although CFS is much less common in children than in adults, children can develop the illness, particularly during the teen years.

CFS is marked by extreme fatigue that has lasted at least six months; is not the result of ongoing effort; is not substantially relieved by rest; and causes a substantial reduction in daily activities. In addition to fatigue, CFS includes eight characteristic symptoms: post-exertional malaise (relapse of symptoms after physical or mental exertion); un-refreshing sleep; substantial impairment in memory/concentration; muscle pain; pain in multiple joints; headaches of a new type, pattern or severity; sore throat; and tender neck or armpit lymph nodes. Symptoms and their consequences can be severe. CFS can be as disabling as multiple sclerosis, lupus, rheumatoid arthritis, congestive heart failure and similar chronic conditions. Symptom severity varies from patient to patient and may vary over time for an individual patient.

There are no physical signs that identify CFS and there are no diagnostic laboratory tests for CFS. People who suffer the symptoms of CFS must be carefully evaluated by a physician because many treatable medical and psychiatric conditions are hard to distinguish from CFS. Common conditions that should be ruled out through a careful medical history and appropriate testing include mononucleosis, Lyme disease, thyroid conditions, diabetes, multiple sclerosis, various cancers, depression and bipolar disorder. Research conducted by the Centers for Disease Control and Prevention (CDC) indicates that less than 20% of CFS patients in this country have been diagnosed.

CFS affects each individual differently. Some people with CFS remain homebound and others improve to the point that they can resume work and other activities, even though they continue to experience symptoms. Recovery rates for CFS are unclear. Improvement rates varied from 8% to 63% in a 2005 review of published studies, with a median of 40% of patients improving during follow-up.

Some patients with CFS may also exhibit symptoms of orthostatic instability, in particular frequent dizziness and light-headedness. Depending on severity and clinical judgment, these patients should be referred for evaluation by a cardiologist or neurologist. Specific treatment for orthostatic instability should only be initiated following confirmed diagnosis and by clinicians experienced in evaluating therapeutic results and managing possible complications. Treatments for orthostatic problems include volume expansion for CFS patients who don't have heart or blood vessel disease. If symptoms don't improve with increased fluid and salt intake, prescription medications and support hose can be prescribed.

Nutritional supplements and vitamins are frequently used by people with CFS for symptom relief.

Treatment and/or Alleviation of CFS

In one embodiment of the present invention, a patient with CFS can be treated with an oral hydration drink comprising the NSP complex as described herein. As a non-limiting example, the drink is a 500 ml drink comprising a concentration of 400-500 mg of sodium chloride (NaCl), 50-100 mg of potassium chloride (KCl), and 25-50 mg magnesium chloride (MgCl), wherein the compounds are a part of the NSP complex solution of the present invention. The NSP complexes of the drink can further comprise 10-15 g of desired proteins although not necessary. The NSP complexes of the drink can further comprise various carbohydrates if desired; however, the drink without the proteins and carbohydrates has a caloric value of 20-30 calories. Thus, if low caloric intake is a concern, then the drink mixture could be adjusted accordingly to acquire the desired level of nutritional supplements, vitamins and/or natural components such as herbs and extracts. Components, volumes and concentrations of this embodiment can be adjusted for each individual patient. The drink may be maintained in the concentrated shelf-stable NSP complex solution for ready dilution when desired or may be formed by reconstituting the dehydrated form as described herein.

In should be understood that the treatment is not limited to oral hydration drink, but can also be administered via intravenous fluids that comprise the components as listed above in forms suitable for intravenous delivery. Fluid delivery via injection should be adjusted for the individual in need thereof.

Vasodepressor Carotid Sinus Syndrome (VCSS)

New approaches to the treatment and prevention of neurally mediated reflex (neurocardiogenic) syncope related disorders are needed. In the United States millions of people are affected by this disorder. Neurally mediated reflex syncope (sometimes referred to as neurocardiogenic syncope), encompasses a group of disorders of which the best known and most frequently occurring forms are the vasovagal (or common) faint, and VCSS. Treatment of most neurally mediated reflex faints is shifting from reliance on various drugs to greater emphasis on education and non-pharmacologic therapy. Initial management should include counseling of patients regarding recognition of early warning symptoms, and avoidance of precipitating factors. However, when initial management is ineffective, volume expansion with salt tablets or electrolyte-containing beverages are important.

In VCSS dizziness, pre-syncope or syncope may be precipitated by any maneuver which causes mechanical stimulation of the carotid sinus—such as turning the head, looking up, or wearing tight collars. The carotid sinus is a dilated portion of one of the major arteries supplying blood to the head. The sinus has nerve endings and acts as a pressure detector feeding back information to the vasomotor center—an area in the brain stem that controls blood pressure and heart rate. Carotid Sinus Syndrome is diagnosed when typical pre-syncopal or syncopal symptoms accompany carotid sinus massage. Of all cases, 26 percent of unexplained syncope cases are found to have Carotid Sinus Syndrome. Carotid Sinus Syndrome is rare in individuals under 50 year's age. Interestingly, 80% of fallers found to have carotid sinus syndrome are amnesic for witnessed associated loss of consciousness. The individual will simply report a fall—an important point in history taking. Prodromal symptoms are more common with vasodepressor carotid sinus syndrome, making it easier to take appropriate action.

Volume maintenance can control VCSS, preventing syncopal episodes by maintaining adequate central volume. An individual without another cardiovascular disease should increase salt intake and drink more fluids containing electrolytes to maintain the volume.

Treatment and/or Alleviation of VCSS

In one embodiment of the present invention, a patient suffering symptoms of or diagnosed with VCSS or related conditions can be treated with an oral hydration drink comprising the NSP complexes as described herein. As a non-limiting example, the drink can be a 500 ml drink comprising a concentration of 400-500 mg of sodium chloride (NaCl), 50-100 mg of potassium chloride (KCl), and 25-50 mg magnesium chloride (MgCl), wherein the compounds are a part of the NSP complex solution of the present invention. The NSP complexes of the drink can further comprise 10-15 g of desired proteins although not necessary. The NSP complexes of the drink can further comprise various carbohydrates if desired; however, the drink without the proteins and carbohydrates has a caloric value of 20-30 calories. Thus, if low caloric intake is a concern, then the drink mixture could be adjusted accordingly to acquire the desired level of nutritional supplements, vitamins and/or natural components such as herbs and extracts. Components, volumes and concentrations of this embodiment can be adjusted for each individual patient. The drink may be maintained in the concentrated shelf-stable NSP complex solution for ready dilution when desired or may be formed by reconstituting the dehydrated form as described herein.

It should be understood that the treatment is not limited to oral hydration drink, but can also be administered via intravenous fluids that comprise the components as listed above in forms suitable for intravenous delivery. Fluid delivery via injection should be adjusted for the individual in need thereof.

Manufacture of the products and their uses within the scope of this invention lends itself to many compounds with therapeutic biological affects. One of skill in the art will see the applicability of the present invention in administration of the present products to any surface of living organisms where there is a particular bio-affecting compound desired for delivery to and/or through that surface.

Although the foregoing description is directed to the preferred embodiments of the present invention, it is noted that other variations and modifications will be apparent to those skilled in the art, and may be made without departing from the spirit or scope of the invention. Moreover, features described in connection with one embodiment of the invention may be used in conjunction with other embodiments, even if not explicitly stated above.

What is claimed is:

1) A method of preparing a phospholipid delivery system for use in encapsulating bio-affecting compounds, said method comprising solubilizing a heterogeneous phospholipid mixture into a suitable organic solvent to form a concentrated formulation of phospholipids; wherein the phospholipids comprise a negatively charged phospholipid species.

2) A phospholipid delivery system for use in encapsulating bio-affecting compounds, comprising a heterogeneous phospholipid mixture, including negatively charged phospholipid species, in a suitable organic solvent.

3) A method of preparing an intermediary nanoscale particle complex (NSP) comprising the steps of:
i) solubilizing a heterogeneous phospholipid mixture in a first quantity of a non-aqueous solvent appropriate to completely solubilize phospholipids into an optically clear solution of a phospholipid delivery system;
ii) solubilizing concentrated bio-affecting compounds in an aqueous solution in an amount sufficient to produce a shelf stable solution of bio-affecting compounds to be encapsulated by the phospholipid delivery system of (i); and
iii) mixing the products of steps (i) and (ii) to produce an intermediary shelf-stable NSP complex encapsulating bio-affecting compounds, wherein the complex is in concentrated form.

4) A shelf-stable NSP complex in a concentrated form comprising:
i) a phospholipid delivery system comprising a heterogeneous phospholipid mixture, including negatively charged phospholipid species, in a suitable organic solvent;
ii) at least one concentrated bio-affecting compound in an aqueous solution; and
wherein (i) and (ii) form the concentrated intermediary shelf-stable NSP complex with the at least one bio-affecting compound encapsulated in the phospholipid delivery system.

5) A method of making an NSP complex for administration comprising the steps of:
i) solubilizing a heterogeneous phospholipid mixture in a first quantity of a non-aqueous solvent appropriate to completely solubilize phospholipids into an optically clear solution;
ii) solubilizing concentrated bio-affecting compounds in an aqueous solution in an amount sufficient to produce a shelf stable solution of bio-affecting compounds to be encapsulated by the phospholipid mixture of (i);
iii) mixing products of steps (i) and (ii) to produce a shelf stable intermediary concentrated NSP complex; and
iv) producing finished NSP complex with encapsulated bio-affecting compounds by dilution of product from step (iii) into a suitable amount of aqueous solution.

6) The method of claim 3, 4 or 5, wherein the encapsulated at least one bio-affecting compound is partially separated from bio-affecting compounds not encapsulated.

7) The method of claim 6, where separation is by centrifugation, precipitation, or exclusion chromatography.

8) The method of claim 3, 4 or 5, wherein less than 50% of the bio-affecting compound is not encapsulated in a phospholipid delivery system.

9) The method of claim 3, 4 or 5, wherein less than 20% of the bio-affecting compound is not encapsulated in a phospholipid delivery system.

10) The method of claim 3, 4 or 5, wherein less than 10% of the bio-affecting compound is not encapsulated in a phospholipid delivery system.

11) The method of claim 3, 4 or 5, wherein less than 5% of the bio-affecting compound is not encapsulated in a phospholipid delivery system.

12) An NSP complex for administration comprising a phospholipid encapsulated bio-affecting compound for administration to a subject in need thereof.

13) A phospholipid encapsulated bio-affecting compound composition manufactured by the steps of:
i) solubilizing a heterogeneous phospholipid mixture in a first quantity of a non-aqueous solvent appropriate to completely solubilize phospholipids into an optically clear solution of a phospholipid delivery system;
ii) solubilizing concentrated bio-affecting compounds in an aqueous solution in an amount sufficient to produce a shelf stable solution of bio-affecting compounds to be encapsulated by the phospholipid delivery system of (i); and
iii) mixing the products of steps (i) and (ii) to produce an intermediary shelf-stable phospholipid encapsulated bio-affecting compound composition, wherein the composition is in concentrated form.

14) A phospholipid encapsulated bio-affecting compound composition for administration to a subject in need thereof, manufactured by the steps of:
i) solubilizing a heterogeneous phospholipid mixture in a first quantity of a non-aqueous solvent appropriate to completely solubilize phospholipids into an optically clear solution;
ii) solubilizing concentrated bio-affecting compounds in an aqueous solution in an amount sufficient to produce a shelf stable solution of bio-affecting compounds to be encapsulated by the phospholipid mixture of (i);
iii) mixing products of steps (i) and (ii) to produce an intermediary shelf-stable phospholipid encapsulated bio-affecting compound composition, wherein the composition is in concentrated form; and
iv) producing a phospholipid encapsulated bio-affecting compound composition for administration by diluting the product from step (iii) into a suitable amount of aqueous solution.
